(19) United States
(12) Patent Application Publication
(10) Pub. No.: US 2007/0041993 A1
(43) Pub. Date: Feb. 22, 2007

(54) Compositions and methods for symptomatic relief

(76) Inventors: Terri Lee Holcomb-Halstead, Grand Terrace, CA (US); Zhenguo Wang, Grand Terrace, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/558,795

(22) PCT Filed: May 26, 2004
(86) PCT No.: PCT/US04/16568

§ 371 Date: Aug. 8, 2006

(30) Foreign Application Priority Data
May 29, 2003 (WO) PCT/US03/17131

(51) Int. Cl. A61K 36/258; A61K 36/254; A61K 36/06; A61K 36/48; A61K 36/232; A61K 36/72

(52) U.S. Cl. 424/195.15; 424/725; 424/728; 424/757

(57) ABSTRACT

An herbal formula provides symptomatic relief for various diseases and especially for patients diagnosed with AIDS/ARC. Particularly preferred formulae include at least part of at least four plants selected from the group consisting of Panax ginseng, Angelica sinensis, Astralagus membranaceus, Ligustrum lucidum, Sophora flavescens, Trichosanthes kirilowii, Agrimonia pilosa, Ganoderma lucidum, Rehmannia glutinosa, Cordiceps sinensis, Oldenlandia diffusea, Isatis spec., Polyporus umbellatus, Pogostemon cablin, Solanum nigrum, Atractylodis macrocephalae, Clematis spec., and Glycyrrhiza spec., and further include at least one of calculus bovis, concha pteriae powder, and borneolum syntheticum.

This application claims the benefit of our copending International patent application with the serial number PCT/US03/17131, filed May 29, 2003, and which is incorporated by reference herein.

### FIELD OF THE INVENTION

Complex compositions and methods for symptomatic relief in patients afflicted with a disease, and especially those afflicted with AIDS/ARC.

### BACKGROUND OF THE INVENTION

Numerous treatments for HIV are known in the art, and among other pharmaceutically active compounds, reverse transcriptase inhibitors have provided significant therapeutic effect to many HIV infected patients. For example, Lamivudine (3TC) or Zidovudine (AZT) have been prescribed for numerous year. However, numerous viral strains have recently emerged with marked resistance against these compounds. To overcome resistance to at least some degree, new nucleoside-type inhibitors may be administered (alone or in combination with other nucleoside-type inhibitors), and exemplary alternative drugs include Stavudine (d4T), Didanosine (ddI), Combivir (a combination of Lamivudine and Zidovudine), and Trizivir (a combination of 3TC, AZT, and Abacavir).

Unfortunately, development of resistance against one nucleoside-type inhibitor may also be accompanied by resistance (to at least some degree) against, another nucleoside-type inhibitor, frequently necessitating a switch to a different class of pharmaceutically active molecules. For example, non-nucleoside-type inhibitors (e.g., Nevirapine, Delavirdine, Efavirenz) are a structurally relatively inhomogeneous group of compounds and are thought to bind in a non-nucleoside pocket of the reverse transcriptases, thereby significantly increasing antiviral efficacy where nucleoside-type inhibitors is employed. While use of non-nucleoside-type inhibitors seems to provide a promising new class of antiviral drugs, several disadvantages still remain. For example, the cost for currently known non-nucleoside-type inhibitors is relatively high, and a single mutation in the viral reverse transcriptases can induce a cross resistance against a wide class of non-nucleoside reverse transcriptase inhibitors.

Alternatively, or additionally, a patient may receive a protease inhibitor (e g., sequinavir, indinavir, nelfinavir, etc.), to reduce or even block viral protein processing within the cell. Unfortunately, certain protease inhibitors are less effective in a single treatment, and are therefore often administered in combination with other anti retroviral agents (e.g., combination of nucleoside reverse transcriptase inhibitors with protease inhibitors, nucleoside reverse transcriptase inhibitors with non-nucleoside reverse transcriptase inhibitors, and even combinations of nucleoside reverse transcriptase inhibitors with non-nucleoside reverse transcriptase inhibitors and protease inhibitors). Unfortunately, combination therapies of protease inhibitors with nucleoside reverse transcriptase inhibitors are often poorly tolerated and frequently lead to premature termination of that therapy.

In still further known treatment options, viral fusion inhibitors (e.g., enfuvirtide) may be employed to attack viral propagation in yet another route. While such approach appears at present relatively promising, cost and availability are often limiting the factors. Moreover, currently available fusion inhibitors must be injected, adding to the inconvenience to the patient. Still further, fusion inhibitors tend to interfere with at least some antiviral medication and are therefore either poorly tolerated and/or limited in their use.

Unfortunately, antiviral treatment in many HIV infected patients often only postpones onset of AIDS and/or ARC, in part owing to the potent medication that ultimately weakens the patients' condition. Moreover, patients tend to develop secondary diseases, including Karposi's sarcoma and opportunistic infections. Once AIDS develops, the immune system's capability to ward off foreign antigens diminishes and bacterial, fungal, and viral infections are unfortunately almost always unavoidable. Besides the obvious lack of sufficient immunity (e.g., development of oral candidiasis, secondary bacterial infection, secondary viral infection, elevated liver enzyme value), AIDS associated symptoms often aggravatesa patients subjective condition. Most typically, ADS associated symptoms include development of and pain associated with peripheral neuropathy and/or Kaposi's sarcoma, fever, cough, night sweat, diarrhea, nausea, lymph swelling, weight loss, loss of appetite, depression, and insomnia.

Deterioration in immunity and increase in AIDS associated symptoms are typically not slowed down or reversed using currently known medication. Therefore, while numerous compositions and methods for treatment of AIDS are known in the art, all or almost all of them suffer from one or more disadvantage. Among other things, despite the relatively high efficacy of known drugs to reduce viral load, most of such drugs will not necessarily bring symptomatic relief. Consequently, there is still a need for improved compositions and methods for symptomatic relief in a patient, and especially in an AIDS patient.

### SUMMARY OF THE INVENTION

The present invention is directed to compositions and methods for symptomatic relief in a patient diagnosed with a disease, and especially in a patient diagnosed with AIDS/ARC. Contemplated composition comprise an herbal component and a non-herbal component, and may further include at least one of a reverse transcriptase inhibitor, a protease inhibitor, a non-nucleoside reverse transcriptase inhibitor, a chelator, and a fusion inhibitor. Alternatively, or additionally contemplated compositions may further include at least one of a mineral supplement, a vitamin supplement, a probiotic supplement, and an enzyme supplement.

It is generally contemplated that a composition for treatment of symptoms associated with a disease includes (1) an herbal combination comprising at least a part of at least four plants selected from the group consisting of Panax ginseng, Angelica sinensis, Astralagus membranaceus, Ligustrum lucidum, Sophora flavescens, Trichosanthes kirilowii, Agrimonia pilosa Ganoderma lucidum, Rehmannia glutinosa, Cordiceps sinensis, Oldenlandia diffusea, Isatis spec., Polyporus umbellatus, Pogostemon cablin, Solanum nigrum, Atractylodis macrocephalae, Clematis spec, and Glycyrrhiza spec., and (2), at least one of calculus bovis, concha pteriae powder, and borneolum syntheticum.

In one especially preferred aspect, the herbal combination comprises at least a part of Panax ginseng, Angelica sinensis, Astralagus membranaceus, Ligustrum lucidum, Sophora flavescens, Trichosanthes kirilowii, Agrimonia pilosa, Ganoderma lucidum, Rehmannia glutinosa, and Cordiceps sinensis, and further comprises at least one of calculus bovis and borneolum syntheticum.

In another especially preferred aspect, the herbal combination comprises at least a part of Cordiceps sinensis, Oldenlandia diffusea, Isatis spec., Polyporus umbellatus, Astralagus membranaceus, Panax ginseng, Pogostemon cablin, Solanum nigrum, Atractylodis macrocephalae, Trichosanthes kirilowii, Clematis spec, Ligustrum lucidum, and Glycyrrhiza spec., and further comprises at least one of calculus bovis and concha pteriae powder.

Therefore, the inventors contemplate a method of providing symptomatic relief to a patient diagnosed with a disease in which contemplated composition are provided at a dosage effective to provide symptomatic relief. In preferred aspects of such methods, the composition is administered to the patient in a daily amount of between 250 mg and 10 g, while especially contemplated diseases include AIDS or ARC. Thus, symptomatic relief particularly includes reduction of pain associated with a neuropathy or Kaposi's sarcoma, and/or reduction of peripheral neuropathy, fever, cough, night sweat, diarrhea, nausea, lymph swelling, weight loss, loss of appetite, oral candidiasis, secondary bacterial infection, secondary viral infection, elevated liver enzyme value, depression, and/or insomnia. Use of such compositions may also improve the general energy level and/or mental clarity.

Consequently, it should further be appreciated that contemplated compositions may be marketed in a manner in which such compositions are provided, and in which information is further provided that the composition provides symptomatic relief to a patient diagnosed with a disease.

Various objects, features, aspects and advantages of the present invention will become more apparent from the following detailed description of preferred embodiments of the invention.

### DETAILED DESCRIPTION

The inventors discovered that various symptoms associated with a disease, and especially symptoms associated with AIDS and ARC can be treated using relatively complex compositions having an herbal component and a non-herbal component. Contemplated compositions and methods demonstrated significant improvement in a patient's well-being and are thought to be effective in a wide range of symptoms associated with a wide range of diseases.

The terms “ARC” and “Aids-Related Complex” are used interchangeably herein and refer to a condition in which antibody tests for an HIV virus in a patient are positive, and in which a patient may exhibit enlarged lymph nodes, fatigue, fever, night sweats, weight loss, and/or unexplained diarrhea, but do not have any of the more serious complications of AIDS.

The terms “AIDS” and “Acquired Immune Deficiency Syndrome” are used interchangeably herein and refer to the presence of at least one of several opportunistic diseases (e.g., Pneumocystis carinii pneumonia, toxoplasmosis, tuberculosis, Karposi's sarcoma, cervical neoplasms, etc.) and/or a CD4 cell count of less than 200 along with serological confirmation (e.g., by antibody determination or PCR) of an infection by an HIV virus.

As also used herein, the term “treatment of symptoms associated with a disease” refers to a treatment that attempts to ameliorate symptoms associated with that disease. Thus, and unless indicated to the contrary, treatment of symptoms is distinct from the treatment of the disease. For example, while contemplated treatments may provide improved energy level, appetite, and/or reduction in pain (e.g., due to peripheral neuropathy or Karposis's sarcoma), the viral load and/or number of T4 helper-cells may not change.

As still further used herein, the term “at least a part of a plant” refers to plant extracts (e.g., prepared by liquid extraction, which may or may not include a step of concentration of the extract), portions of plants (root, leaves, etc.), which may or may not be further processed (e.g., by drying, liquid extraction, etc.), and whole plants, wherein the term “plant” broadly encompasses monocots, dicots, yeasts, fungi, and associated microorganisms.

Furthermore, it should be especially noted that where the specification and/or claims refer to a particular species of a genus, other species of that genus are deemed equivalent so long as the other species contributes to the desired activity of the herbal combination. For example, where the specification refers to Rehmannia glutinosa, other contemplated species of the genus Rehinannia include R. angulata or R. elata. Alternatively, and especially where all species of one genus are contemplated, the genus name may be followed by the term “spec.”. For example, contemplated compositions may include Glycyrrhiza spec., which specifically includes Glycyrrhiza glabra, and Glycyrrhiza lepidota.

As also used herein, the term “about” in conjunction with a numeral refers to a range of ±10% of that numeral, inclusive. For example, where a component is present in an amount of about 10 wt %, a range of 9-11 wt %, inclusive, is contemplated.

Based on the knowledge that certain plants exhibit desirable medical properties, the inventors discovered that combinations of selected plants and/or plant parts (in pulverized and/or extract form) exhibit especially potent pharmaceutical properties when used for treatment of symptoms associated with a variety of diseases. Particularly potent herbal combinations can be prepared from plants, including Abies webbiana, Acacia spec., Acacia Arabia, Agrimonia eupatoria, Ajuga decumbens, Allium cepa, Allium sativum, Aloe vera, Alternanthera philoxeroides or sessiles, Ammi maius, Andographis paniculata, Apium graveolens, Apiurn leptophyllum, Arachis hypogaea, Arctium lappa, Amebia euhcroma, Asparagus racemosus, Astragalus spinosus, Astragalus lentingosis swainsonine, Buchenavia capita, Bryonia cretica ssp., Dioica, Bryonia angustifolia, Camellia theifera, Camellia sinensis, Cedrela toona, Chrysanthemum morifolium, Coffea arabica, Coptis chinesis, Coptis teetoides, Coptis japonica, Coraria nepalensis, Coriandrum sativum, Curcuma longa, Datura metel syn alba, Daucus carota, Echinacea angustiflora and purpurea, Echinacea simulata, Echinacea pallida, Epimedium grandiflorum, Epimedium sagittatum, Epimedium sinense, Epilobium angustifolium, Erigeron Canadensis, Eugenia or Syzigium claviflorum, Fagara xanthox, Foeniculum vulgarel, Gardenia coronaria, Gaultheria trichophylla, Glycine max, Glycyrrhiza labra, Gossypium herbaceum, Heracleum sphondylium, Hypericum perforatum, Hypericum japonicum, Hyssopus officinalis, Jasminum officinale, Lithospermum erythrorhizon, Lonicera japonica, Luffa luffa, Lycopus europaeus, Magnolia officinalis, Mallotus repandus, Mallotus philippinesis, Matricaria chamomil, Matricaria recutitia, Melissa parviflora, Melissa officinalis, Momordica balsamina, Momordica charantia, Narcissus tazetta, Narcissus pseudonarcissus, Oenithera rosea, Paeonia spec., Papaver somniferum, Perilla frutescens, Phyllanthus niruri, Pinus koraicenis, Pinus parviflora, Piper nirgum, Plumeria rubra, Polyantha suberosa, Prunella vulgaris, Prunus bakariensis, Prunus amygdalus, Psoralea corylifolia, Randia dunatorum, Raphanus sativus, Rheum palmatum, Rhus coriaria, Rhus chinesis, Ricinus communis, Rosmarinus officinalis, Salvia miltiorhiza and officinalis, Sambucus ebulus, Saussurea lappa, Scilla griffithii, Scutellaria baicalensis baiealein, Sedum sediforme, Senecio scandenis, Seizecio aereus, Skimmia laureola, Solarium niporum, Swertia franchetiana, Terminalia chebula, Terminalia catappa, Terminalia alata, Thula occidentalis, Trapalaponica spec., Trichosanthes dioica, Trichosanthes kirilowii, Urtica dioica, Viola yeodensis, Woodfordia fruticosa, Woodwardia spec., and Zanoxylum nitidum.

In particularly preferred aspects of the inventive subject matter, herbal combinations are prepared from Radix Glycyrrhizae, Folium Isatidis, Rhizoma Smilacis Glabrae, Flos Lonicerae, Rhizoma Atractylodis Macrocephalae, Fructus Ziziphi Jujubae, Ganoderma Lucidum Japonicum, Polygonum multiflorum Thunb, Radix Paeoniae Alba, Fructus Lycii, Rhizoma Polygonati, Radix Ophiopogonis, Poria, Herba Ecliptae, Fructus Schisandrae, Aconitum camuiichaeli Debx, Radix Morindae Officinalis, Herba Epimedii, Fructus Comi, Radix Isatidis, Radix Ginseng, Radix Angelicae Sinensis, Radix Astragali seu Hedysari, Fructus Ligustri Lucidi, Radix Sophorae Flavescentis, Radix Trichosanthis, Herba Agrimoniae, Ganoderma Lucidum seu Japonicum, Radix Rehmanniae, Cordyceps, Bomeolum Syntheticum, Cordyceps Sinensis, Oldenlandia Diffusae, Natural Indigo, Polyporous Umbellatus, Astragalus Membranaceous, Panax Ginseng, Solanum Nigrum L, Pogostemon Cablin, Atractylodes Macrocephalae, Radix Trichosanthis, Radix Clematidis, Margarita, Ligustrum Lucidum ATT, and Radix Glycyrrhizae. The botanical nomenclature to specific plant parts is employed as commonly used in the art (radix for the root or part thereof; fructus for the fruit or part thereof; flos for the flower or part thereof, etc.).

In further preferred aspects of the inventive subject matter, herbal combinations are prepared from Radix Ginseng, Radix Angelicae Sinensis, Radix Astragali seu Hedysari, Fructus Ligustri Lucidi, Radix Sophorae Flavescentis, Radix Trichosanthis, Herba Agrimoniae, Ganoderma Lucidum seu Japonicum, Radix Rehmanniae, Cordyceps, and Borneolum Syntheticum. Alternatively, the herbal composition may comprise Cordyceps Sinensis, Oldenlandia Diffisae, Natural Indigo, Polyporous Umbellatus, Astragalus Membranaceous, Panax Ginseng, Solanum Nigrum L, Pogostemon Cablin, Atractylodes Macrocephalae, Trichosanthis Radix, Clematidis Radix, Ligustrum Lucidum AIT, and Glycyrrhizae Radix.

It should be recognized that contemplated herbal combinations may be prepared from numerous parts of the plant (or the whole plant), including root, leaf, stem, and flower, including extracts thereof. For example, an herbal combination may be obtained through numerous known procedures, including extraction (e.g., aqueous or non-aqueous solvent, triple point extraction using carbon dioxide, etc), and grinding, etc., and combination of one or more extracts and/or macerates to the final product. Furthermore, it should be appreciated that all extracts, and other plant products may be dried (e.g., freeze dried, distilled, etc.) before use in the combination.

With respect to the non-herbal component of contemplated compositions, it is generally contemplated that all known medically active non-herbal components are suitable. However, especially preferred non-herbal components include at least one of calculus bovis, borneolum syntheticum (isoborneol), and concha pteriae powder. Most preferably, contemplated compositions include the herbal component (typically comprising at least four, and even more typically at least eight plant or plant portions) and the non-herbal component together in a single dosage, however, separate dosage forms are also deemed suitable.

Contemplated composition may therefore be administered in numerous forms and dosages, and most typically as oral composition. For example, where the composition is in powdered form, a capsule, dragee, or tablet may be used. On the other hand, where the composition is in liquid form, the composition may be administered as a syrup, elixir, etc. Furthermore, all parenteral forms of administration are also deemed suitable.

Where a co-administration with supplemental agent (e.g., a reverse transcriptase inhibitor, a protease inhibitor, a non-nucleoside reverse transcriptase inhibitor, a chelator, and/or a fusion inhibitor) is preferred, it should be recognized that various protocols are suitable, and especially contemplated protocols include substantially-simultaneous administration of the supplemental agent (e.g., coadministration in a single tablet), or administration of the supplemental agent (or herbal composition) while there is a measurable concentration of the herbal composition (or supplemental agent) in the patient. For example, it is contemplated that suitable herbal compositions may be orally administered, while the supplemental agent is parenterally administered (e.g., via injection or mucosal presentation). Consequently, the dosage and formulation of contemplated herbal compositions and supplemental agent may vary substantially. However, it is preferred that the extract is administered in approved and/or known dosages and formulations. Similarly, it is preferred that dosages and formulations of appropriate supplemental agent are identical or similar to those known in the art. In still further contemplated aspects, it is preferred that various nutritional supplements (i.e., over-the-counter products) may be provided to a patient, and especially contemplated supplements include mineral supplements (coral minerals, Ca/Mg, etc.), vitamin supplements (e.g., antioxidants), probiotic supplements (e.g., for restoration of intestinal flora), and/or enzyme supplements (e.g., papain, bromelain).

Therefore, the inventors also contemplate a method of providing symptomatic relief to a patient diagnosed with a disease, in which in one step contemplated compositions are provided. In especially preferred aspects, the compositions are formulated for administration to the patient in a daily amount of between 250 mg and 10 g, and even higher (e.g., multiples thereof). For example, symptomatic relief may be provided for diseases including AIDS, ARC, diabetes, various neuropathies, viral infections, and neoplastic diseases (carcinomas, sarcomas, lymphomas).

Among other disease associated symptoms, it is especially contemplated that the symptomatic relief provided by contemplated compositions includes reduction in peripheral neuropathy, fever, cough, night sweat, diarrhea, nausea, lymph swelling, weight loss, loss of appetite, oral candidiasis, secondary bacterial infection, secondary viral infection, elevated liver enzyme value, depression, and insomnia. Further contemplated symptomatic relief also comprises improved energy level and/or mental clarity.

Therefore, further contemplated methods include methods of marketing in which in one step contemplated composition is advertised, offered for sale, or otherwise provided (e.g., sold, mailed, etc.). In another sep, information is provided that the composition provides symptomatic relief to a patient diagnosed with a disease (and especially AIDS/ARC). In such information, reduction of peripheral neuropathy, fever, cough, night sweat, diarrhea, nausea, lymph swelling, weight loss, loss of appetite, oral candidiasis, secondary bacterial infection, secondary viral infection, elevated liver enzyme values (e.g., AST, ALT, GGT), depression, and/or insomnia is particularly contemplated.

### EXAMPLES

### Exemplary Extract #1 (ARF-1)

Herbal Component: (1) An aqueous extract is prepared from a combination of cleaned and dried Radix Angelicae Sinensis, Radix Astragali seu Hedysari, Fructus Ligustri Lucidi, Radix Sophorae Flavescentis, Radix Trichosanthis, Herba Agrimoniae, and Ganoderma Lucidum seu Japonicum as follows (exemplary amounts of the individual plant parts for a 1 kg formulation are provided in Table 1 below).

All plant parts are placed into an extracting tank and four parts of water are added to one part of the herbal composition (wt/wt). The mixture is then boiled for approximately two hours and separated from the herbal composition as a first aqueous extract. Water is again added, now at a ratio of three parts water to one part of herbal composition, and boiled for about 1 hour. The so obtained second aqueous extract is combined with the liquid, filtered, and concentrated (typically between 3:1 and 10:1, most typically about 6:1).

(2) A powder is prepared from cleaned Radix Ginseng, Radix Rehmanniae, and Cordyceps. The so obtained powder is dried using a vacuum cold drying process (e.g., freeze-drying), preferably at or below a temperature of about 48 degree Celsius. After drying, the dried plant material is ground into a fine powder and mixed with the extract (1) from the herbal component. This mixture is further dried (e.g., using vacuum cold drying process), and once dried, ground to a powder and sifted.

Non-Herbal Component: Calculus Bovis and Bomeolum Syntheticum are ground to a fine powder and added to the herbal component mixture and mixed well. The so obtained mixed powder is then filled into capsules with each capsule containing about 0.25 grams of powder.

TABLE 1

| | |
|---|---|
| INGREDIENT | KILO |

Herbal Component

| | |
|---|---|
| Radix Ginseng | 0.15 |
| Radix Angelicae Sinensis | 0.45 |
| Radix Astragali seu Hedysari | 0.45 |
| Fructus LigustTi Lucidi | 0.3 |
| Radix Sophorae Flavescentis | 0.3 |
| Radix Trichosanthis | 0.45 |
| Herba Agriunoniae | 0.3 |
| Ganoderma Lucidum seu Japonicum | 0.15 |
| Radix Rehmarmiae | 0.3 |
| Cordyceps | 0.09 |

Non-Herbal Component

| | |
|---|---|
| Calculus Bovis | 0.03 |
| Borneolum Syntheticum | 0.03 |

Thus, in a preferred aspect of the inventive subject matter, it should be appreciated that the approximate raw material to end material ratio for the following ingredients: Radix Angelicae Sinensis; Radix Astragali seu Hedysari; Fructus Ligustri Lucidi; Radix Sophorae Flavescentis; Radix Trichosanthis; Herba Agrimoniae; and Ganoderma Lucidum seu Japonicum is 6:1 (due to the concentration step) while the other ingredients (Radix Ginseng, Calculus Bovis, Radix Rehmanniae, Cordyceps; and Bomeolum Syntheticum) are unconcentrated.

Table 2 below lists ingredients, genus, species, and percentage of each ingredient present (relative to weight of plant (part) before extraction) within the composition.

TABLE 2

| | | | |
|---|---|---|---|
| | | | % OF |
| INGREDIENTS | GENUS | SPECIES | TOTAL |

Herbal Component

| | | | |
|---|---|---|---|
| Radix Ginseng | Panax | ginseng | 5% |
| Radix Angelicae | Angelica | sinensis | 15% |
| Sinensis | | | |
| Radix Astragali | Astragalus | membranaceus | 15% |
| seu Hedysari | | | |
| Fructus Ligustri | Ligustrum | lucidum | 10% |
| Lucidi | | | |
| Radix Sophorae | Sophora | flavescens | 10% |
| Flavescentis | | | |
| Radix Trichosanthis | Trichosanthes | kirilowii | 15% |
| Cordyceps | Cordyceps | sinensis | 3% |
| Herba Agrimoniae | Agrimonia | pilosa | 10% |
| Ganoderma Lucidum | Ganoderma | lucidum | 5% |
| seu Japonicum | | | |
| Radix Rehmanniae | Rehmannia | glutinosa | 10% |

Non-Herbal Component

| | | | |
|---|---|---|---|
| Calculus Bovis | Bos | taurus | 1% |
| | | domesticus | |
| Borneolum Syntheticum | Dryobalanops | aromatica | 1% |

### Exemplary Extract #2 (Tien Xien)

Herbal Component: (1) An aqueous extract is prepared from a combination of cleaned and dried Radix Astragali Membranaceous, Fructus Ligustri Lucidi, leaves of Oldenlandia diffusea and Isatis spec., Polyporus umbellatus, Pogostemon cablin, Solanum nigrum, Atractylodis macrocephalae rhizome, Radix Clemetidi, Radix Trichosanthis, and Radix Glycyrrhiza spec. essentially as described above in (1) of the herbal component of the ARF-1 extract. However, after combination of the aqueous extracts, the extract is reduced to a volume of about 10 ml in which the plant ingredients are present in amounts as indicated in Table 3 below.

(2) A powder is prepared from cleaned Radix Ginseng and Cordyceps sinensis. The so obtained powder is dried using a vacuum cold drying process (e.g., freeze-drying), preferably at or below a temperature of about 48 degree Celsius. After drying, the dried plant material is ground into a fine powder and mixed with the extract (1) from the herbal component.

Non-Herbal Component: Calculus Bovis and Margarite (Concha pteriae) are ground to a fine powder and added to the herbal component mixture and mixed well. The so obtained mixed concoction is then filled into ampoules with each ampoule containing about 10 ml of liquid.

| | |
|---|---|
| INGRDIENT | AMOUNT PER 10 ML |

Herbal Component

| | | |
|---|---|---|
| Cordyceps sinensis | 1150 | mg |
| Oldenlandia diffusae | 425 | mg |
| Isatis spec. (Indigo) | 425 | mg |
| Polyporus umbellatus | 270 | mg |
| Astragalus membranaceous | 255 | mg |
| Panax ginseng | 255 | mg |
| Solanum nigrum L. | 140 | mg |
| Pogostemon cablin | 140 | mg |
| Atractylodes macrocephalae | 125 | mg |
| Trichosanthis radix | 125 | mg |
| Clematidis radix | 125 | mg |
| Glycyrrhizae radix | 65 | mg |
| Ligustrum lucidum AIT. | 65 | mg |

Non-Herbal Component

| | | |
|---|---|---|
| Margarite | 65 | mg |
| Calculus Bovis | 140 | mg |

It should be appreciated that the percentages and amounts represented here may vary depending on various factors, including pureness of herbal compositions available, severity of symptoms, weight and age of patient, and various other factors. Therefore, the relative weight ratios of the herbal component may vary considerably, and it should be understood that the above exemplary formulations are provided only for guidance. In other contemplated aspects, the non-herbal composition may also vary considerably, as well as the weight ratio between the herbal component and the non-herbal component.

It should further be appreciated that alternative extraction processes are also deemed suitable, and especially preferred extraction processes include extractions with aqueous solvents, mixtures of aqueous solvents with non-aqueous solvents, and even non-aqueous solvents. Furthermore, where suitable, liquid extracts may also,be fractionated using a two-solvent system, or chromatographic methods (e.g., size exclusion, ion exchange, hydrophobic interaction, etc.) to enrich one or more extracts in a desirable component (or to remove undesirable components). Still further, it is also contemplated that the extracts may be prepared as dissolved or dispersed liquids, as dried preparations, or admixed to an edible carrier.

### Clinical Studies Using ARF-1

Patients were chosen according to the following criteria: Patients tested positive to HIV using ELISA and/or Western Blot, T-lymphocyte count was abnormal (e.g., T4 to T8 ratio was below 1, the number of T4 cells was below 400/mm³), pregnancy was excluded, and all patients agreed to use treatment with herbal medicine. Total number of patients was 10, with 5 men and 5 women. Average age was about 31 years, with the oldest being 35, and the youngest being 25. According to the CDC classification, the 10 cases all had HIV associated symptoms (4 diagnosed as ARC and 6 diagnosed as AIDS). Two capsules of the ARF-1 composition as described above were given three times a day over a course of 3 months.

Treatment was evaluated using ELISA test to indicate the presence of the HIV virus, flow cytometry (FACSCAN) every three months before or after treatment to calculate the rate of T4 to T8, and the quantity of T4 (in cubic centimeters) as the index to measure the immune function of treated patients. Diagnostic records were prepared, including physical sign or appearance, appearance of the tongue, and pulse condition. The following symbols were used: evident: (+++); general (++); slight (+); fluctuant ±); and negative (−). A numeric score was based on the conversion table shown below:

| NUMERIC SCORE | SYMBOL |
|---|---|
| 6 | +++ |
| 4 | ++ |
| 2 | + |
| 1 | ± |
| 0 | − |

Criteria for Immune Function: The ratio of T4 to T8 and number T4 cells was observed and used as criteria to measure the state of the immune system: if both of the items increase or either of them increase without a change in the other, there is an improvement in immune function; if both of the items decrease or either of them decrease without a change in the other, the immune system worsens; if neither of the items change, then there is no change in the immune system; if one of them increases while the other decreases, the immune system is judged according to the increase or decrease in the quantity of T4 cells.

Criteria For Curative Effect: Curative Effect was categorized as (a) recovery, (b) evident effect, (c) with effect, and (d) no effect.

(a) Recovery: If serum antibody is negative (with the method of ELISA and WB) and the testing of antigen P24 and PCR is also negative, the immune function recovers to normal (ratio of T4 to T8>1 and the number of T4 cells are beyond 800/mm3). There are no symptoms, no physical signs and no opportunity infections.

(b) Evident effect: If Antigen HIV appears negative or positive, the immune function improves dramatically (ratio of T4 to T8>1, but the number of T4 cell is 400/mm³), the opportunistic infections were basically eliminated, and the symptoms and physical signs recover fundamentally back to normal.

(c) With effect: If Antibody HIV appears positive, the immune function is improving (ratio of T4 to T8>0.2, the quantity of T4 cells is above 400/mm³). Opportunistic infections are receding, and the symptoms and physical signs are substantially improved.

(d) No effect: If there are no dramatic changes on antibody HIV including index of immune function, there is a decrease of immune function in the treatment.

### Results Using ARF-1

The weight of patients in the treatment class remained relatively stable, with a fluctuation of approximately 2 kg. Clinical symptoms generally improved and especially included tiredness, fever, cough, shortness of breath, chest aches, diarrhea, anorexia, night sweat, and lymphadenectasis. All ten patients appeared to be in good condition and reported an average score in improvement of symptoms of 4.6.

The ratio of T4 to T8 of the 10 patients before and after the treatment shows 3 patients who have increased ratios, while the other 7 show no evident change. The numbers of T4 cells of the 3 patients increased. According to the study, the immune function is divided into three conditions after treatment such improvement, no change and worse. In this study, 3 cases improved, 3 cases showed no change and 4 cases became worse. Thus, while contemplated compositions have at least some effect on the physiology and/or immune system, symptomatic relief was significantly improved, raising the quality of life.

Similar results were reported in a second trial (data not shown) in which EGTA was given as a suppository with moderate to great improvement in symptomatic treatment and with inconclusive results with respect to improvements in immune function.

### Clinical Studies Using Tien Xien

Patients were chosen according to the following criteria: Patients tested positive to HIV using ELISA and/or Western Blot, T-lymphocyte count-was abnormal (e.g., T4 to T8 ratio was below 1, the number of T4 cells was below 400/mm³), pregnancy was excluded, and all patients agreed to use treatment with herbal medicine. Patients were on various HAART (Highly Active Anti-Retroviral Therapy) treatment and continued such treatment throughout the observation. Depending on severity of symptoms and overall condition, patients were advised to take two Tien Xien vials (10 ml each) per day.

### Results using Tien Xien

The test results are reported on a per patient basis and clearly demonstrate a substantial improvement in overall well being and reduction of HIV-associated symptoms.

Patient (1) (Taking one vial of Tien Xien/day): Quality of life: Reports ongoing improvement in quality of life indicators including increased mental clarity, decreased fatigue, increased ability to exercise. Any difficulty sleeping resolved many months ago during previous trial. Peripheral neuropathy: Symptoms of this are a sensation of inflamed feet by the end of the day. These symptoms have continued to decrease. Patient began to report normal sensation in his feet. Diarrhea: Over the last month diarrhea has decreased or almost stopped. No change in HAART reported.

Patient (2) (Tien Xien discontinued before end of observation. Normally takes two Tien Xien vials per day). Quality of life: Reports decrease in quality of life indicators since the flu in May including poor appetite, weight loss of 8 pounds, fatigue, depression and sleeping only 5 hours per nite. He has new, moderate pain over the right subcostal margin. Patient also reports quitting his job due to the recent illness. Peripheral neuropathy: Reports a continuation of decreasing pain and increasing normal sensations in his feet. Diarrhea started with flu symptoms and has decreased to mild. Restarted HAART, started Welbutrin and Rimron.

Patient (3) (Taking 3 vials of Tien Xien per day): No change in medication, lab history, and weight. Slight decrease in quality of sleep (waking up frequently but able to return to sleep quickly). Energy level: no change. Pain: The rib cage pain has resolved. Has increased discomfort while sitting due to loss of muscle mass in the buttocks. Peripheral neuropathy: Pain is a 4/10 and off and on with decreased swelling in the left ankle and foot that is chronic since severe KS. Stools: They have darkened to a more normal color from clay colored, has 3 formed stools a day, had 1-2 days of diarrhea last month. Work/activity: very active and continues to increase the workload.

Patient (4) (Taking six vials of Tien Xien per day, 2 vials each morning, noon, night): No weight change and good appetite. Pain/peripheral neuropathy: Leg or hand cramps of short duration at night, no pain in the feet, no episodes of dropping things as previously. Stools: normal stools every three days.

Patient (5) (Taking 3 vials of Tien Xien): No change in weight, with improved sleep and energy level. Pain: Has increased pain in the buttocks when sitting. Peripheral neuropathy showed improvement. Stools: No diarrhea, normal color, consistency and frequency. Work/activity: More active over last month. Karposi's sarcoma: Continues to fade.

Patient (6) (Takes three Tien Xien vials at 2 AM and again at 2 PM): No change in medication, gained weight (6 pounds). Sleep: Good, 7-8 hours per night; Energy level: Good; Pain: Only some sinus pressure; Peripheral neuropathy: A few leg cramps/muscle spasms for one to one and a half weeks, hands much improved; Stools: A normal stool every 3 days, has had some bloating.

Thus, specific embodiments and applications of improved compositions and methods for symptomatic relief have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A composition for treatment of symptoms associated with a disease comprising:
a combination comprising at least a part of at least four plants selected from the group consisting of Panax ginseng, Angelica sinensis, Astralagus membranaceus, Ligustrum lucidum, Sophora flavescens, Trichosanthes kirilowii, Agrimonia pilosa, Ganoderma lucidum, Rehmannia glutinosa, Cordiceps sinensis, Oldenlandia diffusea, Isatis spec., Polyporus umbellatus, Pogostemon cablin, Solanum nigrum, Atractylodis macrocephalae, Clematis spec, and Glycyrrhiza spec.; and
further comprising at least one of calculus bovis, concha pteriae powder, and borneolum syntheticum.

2. The composition of claim 1 wherein the combination comprises at least parts of at least eight plants.

3. The composition of claim 2 wherein the combination comprises at least a part of Panax ginseng, Angelica sinensis, Astralagus membranaceus, Ligustrum lucidum, Sophora flavescens, Trichosanthes kirilowii, Agrimonia pilosa, Ganoderma lucidum, Rehmannia glutinosa, and Cordiceps sinensis.

4. The composition of claim 3 comprising at least one of calculus bovis and borneolum syntheticum.

5. The composition of claim 4 wherein the at least part of Panax ginseng is present between 1 wt % and 10 wt %, Angelica sinensis is present between 10 wt % and 20 wt %, Astralagus membranaceus is present between 10 wt % and 20 wt %, Ligustrum lucidum is present between 5 wt % and 15 wt %, Sophora flavescens is present between 5 wt % and 15 wt %, Trichosanthes kirilowii is present between 10 wt % and 20 wt %, Agrimonia pilosa is present between 5 wt % and 15 wt %, Ganoderma lucidum is present between 1 wt % and 10 wt %, Rehmannia glutinosa is present between 5 wt % and 15 wt %, and Cordiceps sinensis is present between 1 wt % and 8 wt %.

6. The composition of claim 4 wherein the at least part of Panax ginseng is present at about 5 wt %, Angelica sinensis is present at about 1 5 wt %, Astralagus membranaceus is present at about 15 wt %, Ligustrum lucidum is present at about 10 wt %, Sophora flavescens is present at about 10 wt %, Trichosanthes kirilowii is present at about 15 wt %, Agrimonia pilosa is present at about 10 wt %, Ganoderma lucidum is present at about 5 wt %, Rehmannia glutinosa is present at about 10 wt %, and Cordiceps sinensis is present at about 3 wt %.

7. The composition of claim 2 wherein the combination comprises at least a part of Cordiceps sinensis, Oldenlandia difflisea, Isatis spec., Polyponis umbellatus, Astralagus membranaceus, Panax ginseng, Pogostemon cablin, Solanum nigrum, Atractylodis macrocephalae, Trichosanthes kirilowii, Clematis spec, Ligustrum lucidum, and Glycyrrhiza spec.

8. The composition of claim 6 comprising at least one of calculus bovis and concha pteriae powder.

9. The composition of claim 8 wherein the at least part Cordiceps sinensis is present between 25 wt % and 35 wt %, Oldenlandia diffusea is present between 10 wt % and 15 wt %, Isatis spec. is present between 10 wt % and 15 wt %, Polyporus umbellatus is present between 5 wt % and 10 wt %, Astralagus membranaceus is present between 5 wt % and 10 wt %, Panax ginseng is present between 5 wt % and 10 wt %, Pogostemon cablin is present between 2 wt % and 8 wt %, Solanum nigrum is present between 2 wt % and 8 wt %, Atractylodis macrocephalae is present between 2 wt % and 8 wt %, Trichosanthes kirilowii is present between 2 wt % and 8 wt %, Clematis spec. is present between 1 wt % and 5 wt %, Ligustrum lucidum is present between 1 wt % and 4 wt %, and Glycyrrhiza spec. is present between 1 wt % and 4 wt %.

10. The composition of claim 8 wherein the at least part Cordiceps sinensis is present at about 32 wt %, Oldenlandia diffuisea is present at about 12 wt %, Isatis spec. is present at about 12 wt %, Polyporus umbellatus is present at about 7 wt %, Astralagus membranaceus is present at about 7 wt %, Panax ginseng is present at about 7 wt %, Pogostemon cablin is present at about 4 wt %, Solanum nigrum is present at about 3.5 wt %, Atractylodis macrocephalae is at about 3.5 wt %, Trichosanthes kirilowii is at about 3.5 wt %, Clematis spec. is present at about 2.1 wt %, Ligustrurn lucidum is present at about 1.8 wt %, and Glycyrrhiza spec. is present at about 1.8 wt %.

11. The composition of claim 1 further comprising at least one of a reverse transcriptase inhibitor, a protease inhibitor, a non-nucleoside reverse transcriptase inhibitor, a chelator, and a fusion inhibitor.

12. The composition of claim 1 further comprising at least one of a mineral supplement, a vitamin supplement, a probiotic supplement, and an enzyme supplement.

13. A method of providing symptomatic relief to a patient diagnosed with a disease, comprising providing a composition according to claim 1 at a dosage effective to provide symptomatic relief.

14. The method of claim 13 wherein the composition is administered to the patient in a daily amount of between 250 mg and 10 g.

15. The method of claim 13 wherein the disease is AIDS or ARC, and wherein the symptomatic relief comprises reduction of pain associated with a neuropathy or Kaposi's sarcoma.

16. The method of claim 13 wherein the symptomatic relief comprises reduction in at least one of peripheral neuropathy, fever, cough, night sweat, diarrhea, nausea, lymph swelling, weight loss, loss of appetite, oral candidiasis, secondary bacterial infection, secondary viral infection, elevated liver enzyme value, depression, and insomnia.

17. The method of claim 13 wherein the symptomatic relief comprises at least one of improved energy level and mental clarity.

18. A method of marketing a product comprising providing a composition according to claim 1, and providin ginformation that the composition provides symptomatic relief to a patient diagnosed with a disease.

19. The method of claim 18 wherein the disease is AIDS or ARC.

20. The method of claim 18 wherein the symptomatic relief comprises reduction in at least one of peripheral neuropathy, fever, cough, night sweat,. diarrhea, nausea, lymph swelling, weight loss, loss of appetite, oral candidiasis, secondary bacterial infection, secondary viral infection, elevated liver enzyme value, depression, and insomnia.
